(19) United States
(12) Patent Application Publication
(10) Pub. No.: US 2026/0355379 A1
(43) Pub. Date: Nov. 12, 2026

(54) BIOLOGICALLY DEGRADABLE SUPERABSORBER

(71) Applicant: Stockhausen Superabsorber GmbH, Krefeld (DE)

(72) Inventors: Markus Henn, Gelsenkirchen (DE); Christoph Loick, Duisburg (DE); Rainer Teni, Moers (DE); Marc Hager, Haguenau (FR)

(21) Appl. No.: 19/138,481

(22) PCT Filed: Dec. 15, 2023
(86) PCT No.: PCT/EP2023/086141

§ 371 Date: Jun. 12, 2025

(30) Foreign Application Priority Data
Dec. 20, 2022 (EP) 22214780.3

(51) Int. Cl. A61L 15/26; C08G 69/10; C08J 3/24

(52) U.S. Cl. CPC A61L 15/26; C08G 69/10; C08J 3/24

(57) ABSTRACT

The present invention relates to a biologically degradable superabsorber based on polyglutamic acid (salt) and the preparation thereof. It was the objective to provide a biologically degradable superabsorber with improved performance. This problem has been solved by a method for preparing a water-absorbing polyglutamic acid (salt), comprising the of: i) providing an aqueous mixture of a non-crosslinked polyglutamic acid (salt) and at least one crosslinker, ii) crosslinking the non-crosslinked polyglutamic acid (salt) to obtain a crosslinked polyglutamic acid (salt)-gel, iii) drying the crosslinked polyglutamic acid (salt)-gel to obtain a dried crosslinked water-absorbing polyglutamic acid (salt), iv) heat treating the dried crosslinked water-absorbing polyglutamic acid (salt) in the presence of at least one facilitator to obtain the water-absorbing polyglutamic acid (salt).

The present invention relates to a biologically degradable superabsorber based on polyglutamic acid (salt) and the preparation thereof.

“Superabsorbent” or “superabsorber” is a common commercial term referring to polymeric particles capable of absorbing huge amounts of water without releasing it under pressure. Other common terms are “super-absorbent material” (SAM), “super-absorbent polymers” (SAP) or “absorbent gelling material” (AGM).

When absorbing water, the superabsorbent swells impetuously; the water is caged within its polymeric network so that the water-laden superabsorbent forms a hydrogel. Likewise water, superabsorbents absorb saline and different kinds of body fluids. Due to this capacity, superabsorbents serve as a key ingredient for personal hygiene products like baby diapers, feminine care products and incontinence articles.

Most of the super-absorbent materials used in hygiene articles today, which are capable of absorbing large quantities of liquids, among them, water and body fluids such as urine, in a short period of time are primarily based upon slightly crosslinked synthetic polymers. These include, for example, polymers and co-polymers based upon acrylic acid or acrylamide, which do traditionally not belong to renewable materials and are insufficiently or not at all biologically degradable resulting in the need for incineration or landfilling.

Attempts to replace the afore-mentioned polymers and co-polymers with biological degradable and hence sustainable polymers have been undertaken. One of the focuses lied upon the use of polysaccharides. A challenge entailed with the raw materials for the production of superabsorbers based upon polysaccharides is, however, that the raw material is frequently water-soluble and must be converted into the water-insoluble form, to be able to use them as superabsorbers in various applications.

For example, EP 0 538 904 A1 and U.S. Pat. No. 5,247,072 describe superabsorbers based upon carboxyalkyl polysaccharides. The process involves a thermal crosslinking step which is very sensitive to small changes of the pH value, the temperature of the reaction duration, absorbers with widely varying absorption properties are obtained with a tendency of shortened storage time duration.

In the processes known from the prior art for crosslinking polysaccharides, however, besides the partially low aging stability, it is observed that the homogeneous crosslinking of the polysaccharides hinders the biodegradability of the absorber, since the accessibility for microorganisms is reduced by the restricted swelling. Furthermore, in the crosslinking reactions known from the prior art, the enzymatic breakdown is inhibited by the additionally introduced substituents [Mehltretter et al., Journal of the American Oil Chemists Society, 47 (1970), pages 522-524].

Due to its bio-based and biodegradable properties, polyglutamic acid (PGA) and its use as superabsorbent material was also investigated. For instance, the International Application WO 2021/242936 A1 reports the manufacture of a superabsorbent based upon polyglutamic acid which may be additionally modified. The superabsorbent, however, shows mediocre performance with respect to common parameters used to assess a superabsorber, such as centrifugal retention capacity (CRC), absorption against pressure (AAP), saline flow conductivity (SFC) and absorption speed (Vortex and FSR), especially after the surface-crosslinking step. As a proper performance regarding said parameters is crucial for commercial success of superabsorbents employed in personal hygiene articles, actual use of polyglutamic acid-based superabsorbents in commercial hygiene articles is not known yet.

It is thus the objective of the present invention to provide a biologically degradable superabsorber with improved performance. In particular, performance regarding centrifugal retention capacity (CRC), absorption against pressure (AAP), saline flow conductivity (SFC) and absorption speed (Vortex and FSR) shall be improved and well-balanced.

This object is solved by a method for producing a water-absorbing polyglutamic acid (salt), comprising the method steps of:
i) providing an aqueous mixture of a non-crosslinked polyglutamic acid (salt) and at least one crosslinker,
ii) crosslinking the non-crosslinked polyglutamic acid (salt) to obtain a crosslinked polyglutamic acid (salt)-gel,
iii) drying the crosslinked polyglutamic acid (salt)-gel to obtain a dried crosslinked water-absorbing polyglutamic acid (salt),
iv) heat treating the dried crosslinked water-absorbing polyglutamic acid (salt) in the presence of at least one facilitator to obtain the water-absorbing polyglutamic acid (salt).

It has been found that—if produced according to the inventive method-superabsorbents based on polyglutamic acid (salt) achieve better performance in regard to demands of users and manufacturers of personal hygiene articles. Since superabsorbents obtained according to the inventive process are originating from polyglutamic acid, they are biodegradable.

“At least one” as used herein relates to one or more, i.e., 1, 2, 3, 4, 5, 6, 7, 8, 9 or more. With respect to a crosslinker, for example, the value relates to the crosslinkers and not to the absolute number of the crosslinker's molecules employed. Unless stated otherwise, “%” refers to “wt. %”.

A contribution to achieving the object is made by a method for producing a water-absorbing polyglutamic acid (salt), comprising the method steps of i) providing an aqueous mixture of a non-crosslinked polyglutamic acid (salt) and at least one crosslinker, ii) crosslinking the non-crosslinked polyglutamic acid (salt) to obtain a crosslinked polyglutamic acid (salt)-gel, iii) drying the crosslinked polyglutamic acid (salt)-gel to obtain a dried crosslinked water-absorbing polyglutamic acid (salt), and iv) heat treating the dried crosslinked water-absorbing polyglutamic acid (salt) in presence of at least one facilitator to obtain the water-absorbing polyglutamic acid (salt) provides for a sustainable and biologically degradable water-absorbing polyglutamic acid (salt).

The employed non-crosslinked polyglutamic acid (salt) of the present invention can be partially neutralized. Preferably, at least 50 mol-%, more preferably at least 70 mol-% and even more preferably at least 80 mol-% of the non-crosslinked polyglutamic acid is neutralized and present as salt. The neutralization can be accomplished by using standard techniques and methods known by the skilled person. For example, an aqueous solution of sodium hydroxide can be used. Suitable cations are lithium, sodium, and potassium and preferably sodium, potassium, and a mixture thereof are selected. The non-crosslinked polyglutamic acid is selected from α-polyglutamic acid and γ-polyglutamic acid, preferably γ-polyglutamic acid is selected. In a preferred embodiment of the present invention, the non-crosslinked polyglutamic acid (salt) has a molecular weight of 200,000 Dalton to 3,000,000 Dalton, preferably 500,000 Dalton to 2,000,000 Dalton, more preferably 700,000 Dalton to 1,100,000 Dalton.

“Aqueous mixture”, as used herein, refers to a mixture comprising at least 40 wt. %, preferably at least 50 wt. %, more preferably at least 60 wt. %, and even more preferably 70 wt. % water based on total weight of the aqueous mixture.

The at least one crosslinker used in step i) is selected from the group consisting of glycidyl ether such as diglycidyl ether, triglycidyl ether, polyglycidyl ether containing 3 or more epoxy groups, diglycerol tetraglycidyl ether, dipentaerythritol tetraglycidyl ether, pentaerythritol polyglycidyl ether, sorbitol polyglycidyl ether, isosorbide glycidyl ethers, polyglycerol-3-glydidyl ether, or other aliphatic polyfunctional epoxides, 1,4-butanediol diglycidyl ether, 1,6-hexanediol diglycidyl ether, propylene glycol diglycidyl ether, di propylene glycol diglycidyl ether, trimethylolpropane triglycidyl ether, poly propylene glycol diglycidyl ether, polyglycidyl ethers of alkanepolyols, polyglycidyl ethers of poly(alkylene glycols), biobased sorbitol glycidyl ether e.g. ERISYS® GE-61 from CVC Thermoset Specialties, any type of cyclic and aromatic poly glycidyl ethers, or a combination of more than one crosslinker of the class of glycidyl ether. The employed at least one crosslinker can be based on the carbodiimide chemistry and can be selected from 1-ethyl-3-(3-dimethylamino propyl) carbodiimide hydrochloride, dicyclohexyl carbodiimide or other carbodiimides containing compounds or a combination of carbodiimide functionality containing crosslinker with other crosslinkers e.g. crosslinker of the group of glycidyl ethers saccharides such as glucose, maltotriose or cyclodextrin in the presence of water-soluble carbodiimide containing compound. Further crosslinkers can be used such as water-soluble chitosan, polyethylene glycol or other organic poly alcohols, aryl azide or diazirine or other photoreactive chemical hetero bi-functional crosslinking agents acting as receptor-ligand-interaction complexes by a 2-step activation, ethylene glycol diglycidyl ether, polyethylene glycol diglycidyl ether and 1,4-butanediol diglycidyl ether. Preferably, a crosslinker selected from the group consisting of ethylene glycol diglycidyl ether, Poly(ethylenglycol) diglycidylether and 1,4-butanediol diglycidyl ether is used. In case Poly(ethylenglycol) diglycidylether is used, the average molecular weight of such crosslinker may be below 2000 g/mol, for instance 500 g/mol.

In an embodiment of the invention, the aqueous mixture contains a water-soluble polymer which can be selected from the group consisting of a water-soluble polymer derived from the ethylenically unsaturated monomers containing acid groups defined in the International Application WO 2004/037903 A2, which is introduced herewith by reference and thus forms part of the disclosure. The polymers can be based on the acrylic acid and methacrylic acid, acrylamides, and methacrylamides. Preferred (meth)acrylamides are, in addition to acrylamide and methacrylamide, alkyl-substituted (meth)acrylamides or amino alkyl-substituted derivatives of (meth)acrylamide, such as N-methylol (meth)acrylamide, N,N-dimethylamino (meth)acrylamide, dimethyl-(meth) acrylamide or diethyl(meth)acrylamide. Possible vinylamides are, for example, N-vinylamides, N-vinylformamides, N-vinylacetamides, N-vinyl-N-methylacetamides, N-vinyl-N-methyl formamides and vinylpyrrolidone. Further, the water-soluble polymer can be based on alkoxy-polyalkylene oxide (meth)acrylates, such as methoxy polyethylene glycol (meth)acrylates, acrylic acid esters and methacrylic acid esters, such as methyl (meth)acrylate, ethyl (meth)acrylate, propyl (meth)acrylate or butyl (meth)acrylate. These monomers can also include methyl polyethylene glycol allyl ether, vinyl acetate, styrene and isobutylene.

In addition, further biologically degradable polymers can be employed such as polysaccharides.

Preferably, the aqueous mixture provided in step i) comprises:
a) 10.0 wt. % to 50.0 wt. %, preferably 20.0 wt. % to 40.0 wt. %, and more preferably 30.0 wt. % polyglutamic acid (salt) based on the total weight of the aqueous mixture,
b) 0.1 wt. % to 4.0 wt. %, preferably 0.3 wt. % to 3.0 wt. %, and more preferably 0.5 wt. % to 2.0 wt. % of the at least one crosslinker based on the total weight of the polyglutamic acid (salt),
c) 0.0 wt. % to 10.0 wt. %, preferably 0.0 wt. % to 5.0 wt. %, and more preferably 0.1 to 2.5 wt. % of a water-soluble polymer, and
d) 30.0 wt. % to 90.0 wt. %, preferably 50.0 wt. % to 80.0 wt. %, and more preferably 60.0 wt. % to 70.0 wt. % water.

The crosslinking in step ii) of the method according to the present invention is preferably conducted within 10 min to 50 min, preferably within 20 min to 40 min, and more preferably within 30 min. In a preferred embodiment, step ii) is conducted under atmospheric pressure. In another preferred embodiment, step ii) is conducted under agitation. More preferably, step ii) is conducted under agitation and under atmospheric pressure. Preferably, the crosslinking is conducted in a kneader reactor. The kneader reactor can be a single shaft kneader reactor. In another embodiment, the kneader reactor has at least two kneading shafts. Kneader type reactors are equipped with 10 agitating means for disintegrating the polymerizing material right in the reaction vessel. Using a kneader reactor provides advantages over other common apparatuses such as a belt, as it ensured effective mixing and provides a homogeneous mixture.

In a preferred embodiment, step ii) of the inventive method comprises a first stage and a second stage. The first stage is conducted under maximum agitation and the second stage at 20% to 60%, preferably 30% to 50% and more preferably 40% of the maximum agitation. The time duration of the first stage is between 50% to 82%, preferably 58% to 76%, and more preferably 66% of the total time duration of step ii). Without wishing to be bond to a specific theory, we conclude that the two stages address the changing viscosity as the advancing crosslinking leads to an enhanced viscosity of the material. Thus, by lowering the agitation in the second stage the material is less deteriorated. This method step is preferably conducted at a temperature of at least 80 °C, more preferably at least 90 °C, and even more preferably of at least 100 °C. The afore-mentioned temperature refers to the temperature of the chemical compounds present in step ii) comprising crosslinked polyglutamic acid (salt)-gel and non-crosslinked polyglutamic acid (salt). The reaction vessel is ideally pre-heated to a specific temperature before the aqueous mixture is filled into the reaction vessel. A temperature of 120 °C is preferred.

Using 1,4-BDDGE as crosslinker, a concentration of 33.0 wt. % polyglutamic acid referred to the total weight of the aqueous solution, applying a reaction vessel temperature of −120 °C in step ii) as well as using the first and second stage as described above and a fill grade of at least 50 vol. % is advantageous for the thus obtained crosslinked polyglutamic acid (salt)-gel and also the final product performance.

Alternatively, to a kneader reactor, a reactor without agitating means may be used, for example a belt reactor. Even in such case, crosslinked polyglutamic acid (salt)-gel shall be disintegrated before drying. This enhances the drying efficiency. However, gel obtained from a kneader reactor may be disintegrated before drying as well.

The disintegrating of the gel can, for example, be performed by means of an extruder or a chopper or a mincer or other common apparatuses.

The drying step iii) of the method according to the invention can be accomplished by common apparatuses and techniques. Particularly, the gel can be dried by using a bed drier, a plate belt drier, or a fluidized bed drier or by using a microwave or convection drier. The drying in step iii) is preferably conducted for 5 min to 15 min, more preferably for 10 min at a temperature of between 100 °C to 160 °C, preferably between 115 °C to 145 °C, and more preferably at a temperature of 130 °C. Shorter drying time and/or lower drying temperature leads to wet material, whereas longer drying time and/or higher drying temperature results in undesired CRC loss and AAP loss of the water-absorbing polyglutamic acid (salt).

In case a bed drier is used, the crosslinked polyglutamic acid-gel is preferably turned by 180° after 40% to 60%, preferably after 50% of the drying step is conducted. This ensures even gel drying.

For drying the crosslinked polyglutamic acid-gel in step iii), the hot air stream used in the dryer may have a velocity of 4 m/s to 7 m/s, preferably of 5.5 m/s.

According to a preferred embodiment the dried polyglutamic acid-gel is subject to a grinding step and a sieving step prior to step iv).

Preferably the sieving apparatus used in the sieving step shall have at least two sieves, the first sieve has a mesh size of 850 μm and the second mesh size of 150 μm.

In step iv), the dried crosslinked water-absorbing polyglutamic acid (salt) is heat treated in the presence of at least one facilitator to obtain the water-absorbing polyglutamic acid (salt). “In presence of at least one facilitator” in this context means that the facilitator is brought into contact with the dried crosslinked water-absorbing polyglutamic acid (salt) prior to the heat treatment or over the course of heat treatment, preferably prior to the heat treatment. To that end, means known in the art can be used, for example, spraying the facilitator onto the dried crosslinked water-absorbing polyglutamic acid (salt) and, if needed, by simultaneous or subsequent mixing of the obtained mixture. Ideally, it should be ensured that the facilitator is evenly spread over the dried crosslinked water-absorbing polyglutamic acid (salt). The heat treating of the dried crosslinked water-absorbing polyglutamic acid (salt) in step iv) is conducted at a temperature ranging from 130 °C to 170 °C, preferably at 150 °C for 10 minutes to 90 minutes, preferably for 20 minutes to 60 minutes.

The at least one facilitator according to the invention can be selected from the group consisting of surface-crosslinker, water, and additives.

The surface-crosslinker which can be applied in step iv) is selected from the group consisting of polyols, for example ethylene glycol, polyethylene glycols such as diethylene glycol, triethylene glycol and tetraethylene glycol, propylene glycol, polypropylene glycols such as dipropylene glycol, tripropylene glycol or tetrapropylene glycol, ethylene glycol diglycidyl ether (EGDGE) 1,3-butanediol, 1,4-butanediol, 1,5-pentanediol, 2,4-pentanediol, 1,6-hexanediol, 2,5-hexanediol, 1,4-butandiol diglycidyl ether (BDDGE), glycerine, polyglycerin, trimethylolpropane, polyoxypropylene, oxyethylene-oxypropylene-block copolymer, sorbitan-fatty acid esters, polyoxyethylene sorbitan-fatty acid esters, pentaerythritol, polyvinylalcohol and sorbitol, aminoalcohols, for example ethanolamine, diethanolamine, triethanolamine or propanolamine, polyamine compounds, for example ethylenediamine, diethylenetriamine, triethylene tetraamine, tetraethylene pentaamine or pentaethylene hexaamine, polyglycidyl ether compounds such as ethylene glycoldiglycidyl ether, polyethylene glycoldiglycidyl ether, glycerine diglycidyl ether, glycerine polyglycidyl ether, pentaerythritol polyglycidyl ether, propylene glycoldiglycidyl ether, polypropylene glycoldiglycidyl ether, neopentyl glycoldiglycidyl ether, hexanediolglycidyl ether, trimethylolpropane polyglycidyl ether, sorbitolpolyglycidyl ether, phthalic acid diglycidyl ester, adipinic acid diglycidyl ether, 1,4-phenylenebis(2-oxazoline), glycidol, polyisocyanates, preferably diisocyanates such as 2,4-toluenediioscyanate and hexamethylene diisocyanate, polyaziridine compounds such as 2,2-bishydroxymethylbutanol-tris[3-(1-aziridinyl) propionate], 1,6-hexa-methyl-enediethyleneurea and diphenylmethane-bis-4,4′-N,N′-diethyleneurea, halogen epoxides for example epichloro- and epibromohydrin and alpha-methyl epichlorohydrin, alkylene carbonates such as 1,3-dioxolane-2-one (ethylene carbonate), 4-methyl-1,3-dioxolane-2-one (propylene carbonate), 4,5-dimethyl-1,3-dioxolane-2-one, 4,4-dimethyl-1,3-dioxolane-2-one, 4-ethyl-1,3-dioxolane-2-one, 4-hydroxymethyl-1,3-dioxolane-2-one, 1,3-dioxane-2-one, 4-methyl-1,3-dioxane-2-one, 4,6-dimethyl-1,3-dioxane-2-one, 1,3-dioxolane-2-one, poly-1,3-dioxolane-2-on, polyquaternary amines such as condensation products from dimethylamines and epichlorohydrin. Further preferred surface crosslinkers are in addition polyoxazolines such as 1,2-ethylenebisoxazoline, crosslinkers with silane groups such as γ-glycidooxypropyl trimethoxysilane and γ-aminopropyl trimethoxy silane, oxazolidinones such as 2-oxazolidinone, bis- and poly-2-oxazolidinone and diglycolsilicates. Preferably, the surface-crosslinker is selected from ethylene glycol diglycidyl ether (EGDGE) and 1,4-butandiol diglycidyl ether (BDDGE).

0.10 wt. % to 2.00 wt. % of the surface-crosslinker is regularly used, preferably 0.25 wt. % to 1.40 wt. %, and more preferably 0.50 wt. % to 0.80 wt. % based on the total amount of the dried crosslinked water-absorbing polyglutamic acid (salt).

As facilitator according to the invention, water can also be used in step iv). Typically, 1.0 wt. % to 5.0 wt. %, preferably 2.0 wt. % to 4.0 wt. %, and more preferably 3.0 wt. % water is used based on the total amount of the dried crosslinked water-absorbing polyglutamic acid (salt). Without wishing to be bond to a specific theory, the water assists the heat treating which is beneficial to the performance of the final product, especially to the centrifuge retention capacity and absorption against pressure. In a preferred embodiment, water is used as facilitator in the absence of any other chemical compounds such as organic compounds including, but not limited to, organic solvents, organic surface crosslinkers, organic additives, or organic metal salts, and also in the absence of inorganic compounds such as, but not limited to, metal salts, water-soluble and water-insoluble inorganic compounds. “In the absence” means that the water employed comprises less than 1.0 wt. %, preferably less than 0.5 wt. %, more preferably less than 0.1 wt. % of any other chemical compound with respect to the total amount of the water employed.

Also, as facilitator, an additive can be used for the purpose of improving properties such as gel strength, permeability, processability, odor control, color, etc. For example, these additives may be applied before, during or after the surface crosslinking step. They can also be applied without surface-crosslinking.

The water-absorbing polyglutamic acid (salt) according to the invention may include 0.01 wt. % to 5.00 wt. %, or 0.01 wt. % to 1.00 wt. %, or 0.01 wt. % to 0.05 wt. % of a penetration modifier based on the total weight of the dried crosslinked water-absorbing polyglutamic acid (salt). Eligible examples of penetration modifiers include compounds which alter the penetration depth of the surface-modifying agents into the water-absorbing polyglutamic acid (salt), fiber, film, foam or bead by changing the viscosity, surface tension, ionic character or adhesion of said agents or medium in which these agents are applied. Penetration modifiers may include polyethylene glycols, tetraethylene glycol dimethyl ether, monovalent metal salts, surfactants and water-soluble polymers.

The water-absorbing polyglutamic acid (salt) according to the invention may include 0.01 wt. % to 5.00 wt. %, or 0.01 wt. % to 1.00 wt. %, or 0.01 wt. % to 0.05 wt. % of a multivalent metal salt based on the total weight of the dried crosslinked water-absorbing polyglutamic acid (salt). The multivalent metal salt is preferably water soluble. Examples of metal cations include, but are not limited to, the cations of Al, Fe, Zr, Mg, Ce, and Zn. Preferably, the multivalent metal salt has a valence of +3, with Al(+3) being most preferred. Examples of anions in the multivalent metal salt include halides, sulfates, nitrates, lactates, and acetates, with chlorides, sulfates, acetates, and lactates being preferred, sulfates and lactates being more preferred. Aluminum sulfate and aluminum lactate are examples of multivalent metal salt and are readily commercially available and show great performance. The preferred form of aluminum sulfate is hydrated aluminum sulfate, preferably aluminum sulfate with 12 to 14 water molecules. Mixtures of multivalent metal salts may be employed. The dried crosslinked water-absorbing polyglutamic acid (salt) and multivalent metal salt suitably are mixed by dry blending, or in solution, using means well known to those skilled in the art. With dry blending, a binder may be employed in an amount which is sufficient to ensure that a substantially uniform mixture of the salt and the superabsorbent polymer is maintained. The binder may be water or a low volatile organic compound having a boiling point of at least 150 °C. Examples of binders include water, polyols such as propylene glycol, glycerin and poly(ethylene glycol).

The water-absorbing polyglutamic acid (salt) according to the invention can comprise 0.01 wt. % to 5.00 wt. %, or 0.01 wt. % to 1.00 wt. %, or 0.01 wt. % to 0.05 wt. % of a water-insoluble, inorganic powder based on the total weight of dried crosslinked the water-absorbing polyglutamic acid (salt). Examples of insoluble, inorganic powders include silicon dioxide, silicic acid, silicates, titanium dioxide, aluminum oxide, magnesium oxide, zinc oxide, talc, calcium phosphate, clays, diatomaceous earth, zeolites, bentonite, kaolin, hydrotalcite, activated clays and apatites. The insoluble inorganic powder additive may be a single compound, or a mixture of compounds selected from the above list. An example of silica is SIPERNAT® 22S fumed silica commercially available from Evonik Industries. Prefered particle diameters of the inorganic particles are in the range from 10*10⁻⁹ m to 10*10⁻⁶ m.

In some aspects, the water-absorbing polyglutamic acid (salt) of the present invention comprises 0.01 wt. % to 5.00 wt. %, or 0.01 wt. % to 1.00 wt. %, or 0.01 wt. % to 0.05 wt. % of a polymeric coating, such as a thermoplastic coating, or a cationic coating, or a combination of a thermoplastic coating and a cationic coating based on the total weight of the dried crosslinked water-absorbing polyglutamic acid (salt). In some particular aspects, the polymeric coating is a polymer that may be in a solid, emulsion, suspension, colloidal, or solubilized form, or combinations thereof. Polymeric coatings suitable for this invention may include, but are not limited to, a thermoplastic coating having a thermoplastic melt temperature wherein the polymeric coating is applied to the particle surface coincident with or followed by a temperature of the treated superabsorbent polymer particle at the thermoplastic melt temperature. Examples of thermoplastic polymers include polyolefin, polyethylene, polyester, polyamide, polyurethane, styrene polybutadiene, linear low density polyethylene (LLDPE), ethylene acrylic acid copolymer (EAA), ethylene alkyl methacrylate copolymer (EMA), polypropylene (PP), maleated polypropylene, ethylene vinyl acetate copolymer (EVA), polyester, polyamide, and blends of all families of polyolefins, such as blends of PP, EVA, EMA, EEA, EBA, HDPE, MDPE, LDPE, LLDPE, and/or VLDPE, may also be advantageously employed. In particular aspects, maleated polypropylene is a preferred thermoplastic polymer for use in the present invention. A thermoplastic polymer may be functionalized to have additional benefits such as water solubility or dispersibility. A cationic polymer as used herein refers to a polymer or mixture of polymers comprising a functional group or groups having a potential of becoming positively charged ions upon ionization in an aqueous solution. Suitable functional groups for a cationic polymer include, but are not limited to, primary, secondary, or tertiary amino groups, imino groups, imido groups, amido groups, and quaternary ammonium groups. Examples of synthetic cationic polymers include the salts or partial salts of poly(vinyl amines), poly(allylamines), poly(ethylene imine), poly(amino propanol vinyl ethers), poly(acrylamidopropyl trimethyl ammonium chloride), poly(diallyldimethyl ammonium chloride). Examples of natural-based cationic polymers include partially deacetylated chitin, chitosan, and chitosan salts. Synthetic polypeptides such as polyasparagins, polylysines, and polyarginines are also suitable cationic polymers.

Further, additives that enhance whiteness of the polymer or the long-term color stability such as against darkening, yellowing or browning may also be employed. Such additives are well known in the art and include anti-oxidants, sulfur- and phosphorous-containing compounds, chelating agents, optical brighteners and the like. Preferred additives for color stability are 2-hydroxy-2-sulfonato acetic acid, bisulfites, phosphonates, ethylene diamine tetraaceticacid, ethylene diamine-N,N′-disuccinic acid, diethylene diamine pentaacetic acid, their salts and derivatives, and mixtures thereof.

In some aspects, additional surface additives may optionally be employed with the particulate superabsorbent polymer compositions, including odor-binding or odor controlling substances, such as cyclodextrins, zeolites, inorganic or organic salts, similar materials and the tannins described in the European Patent EP 2 176 325 B1 which are included herein by reference and thus forms part of the disclosure; anti-caking additives, flow modification agents, surfactants, viscosity modifiers, and the like. In addition, surface additives may be employed that perform several roles during surface modifications. For example, a single additive may be a surfactant, viscosity modifier, and may react to crosslink polymer chains. In another aspect, the additional surface additives may comprise a chelating agent. The chelating agent preferably may be selected from organic polyacids, phosphoric polyacids and salts thereof. Preferably, the chelating agent may be selected from nitrilotriacetic acid, ethylene diamine tetraacetic acid, cyclohexane diamine tetraacetic acid, diethylene triamine pentaacetic acid, diethylenetriamine penta(methylene phosphonic acid), ethyleneglycol-bis-(aminoethylether)-N,N,N′-triacetic acid, N-(2-hydroxyethyl)-ethylene diamine-N,N,N′-triacetic acid, triethylene tetraamine hexaacetic acid, tartaric acid, citric acid, imino disuccinic acid, gluconic acid, phosphonates and salts thereof.

The steps of the method according to the invention are conducted in the sequence step i), step ii), step iii), and then step iv).

The present invention also relates to a water-absorbing polyglutamic acid (salt) obtainable by a method described herein. Preferably, the water-absorbing polyglutamic acid (salt) possess a particulate form and/or the water-absorbing polyglutamic acid (salt) possess a particle size distribution of between 150 μm to 850 μm. Preferably, at least 80 wt. %, more preferably at least 90 wt. % of the water-absorbing polyglutamic acid (salt) has a particle size distribution of between 150 μm to 850 μm.

Preferably, the water-absorbing polyglutamic acid (salt) obtainable according to present process achieves at least one of the following performance parameters:
an absorption against pressure of 4.83 kPa (0.7 psi) of more than 9 g/g and less than 30 g/g or less than 26 g/g, preferably less than 25 g/g, as measured as defined in the description;
a saline flow conductivity of more than 0 and less than 150×10⁻⁷·cm³·s·g⁻¹, preferably more than 10 and less than 100×10⁻⁷·cm³·s·g⁻¹ as measured as defined in the description;
a centrifugal retention capacity of more than 20 and less than 46 g/g, preferably more than 25 and less than 42 g/g as measured as defined in the description;
a free swell rate of more than 0.5 and less than 1.5 g·g⁻¹·s⁻¹, preferably 0.7 and less than 1.2 g·g⁻¹·s⁻¹ as measured as defined in the description;
a Vortex of less than 50 and more than 10 seconds, preferably less than 30 and more than 15 seconds as measured as defined in the description.

Even more preferably, the water-absorbing polyglutamic acid (salt) exhibits the AAP and CRC values mentioned above.

Said performance parameters are frequently used for qualifying water-absorbing polymers for use in personal hygiene articles. Cited values are resulting from inventive preparation method.

Yet another object of the present invention is an article comprising the water-absorbing polyglutamic acid (salt) with at least one of the performance parameters or which is obtainable by the method according to the present invention.

Said article is preferably a personal hygiene article such as a diaper, a sanitary towel, or a napkin. Besides that, the article may be a wound covering. Further applications of the inventive water-absorbing polyglutamic acid (salt) include medicine/pharmaceutical applications where it can be used as or in biological glue, dental carriers, bone regeneration, cartilage regeneration, vaccine development scaffolds in tissue engineering, drug delivery system, and biological control agent. Further applications are agricultural, e.g. for improving the soil water availability, the use as biofertilizer, in wastewater treatment, as bio flocculant, in the food industry as thickener, oil reducing agent, flavoring agent, cryoprotectant, and in cosmetics, such as sunscreens, hair growth serums, anti-aging serums, mouth wash, and contact lens care solutions.

### Test Methods

Superabsorbents are usually purchased by manufacturers of personal hygiene products. For purchase decision, the overall performance profile of a superabsorbent is crucial. The required performance profile strongly depends on the type and purpose of the hygiene article. During the last decades, a bunch of performance parameters has been established by the market participants for comparing the suitability of different SAP qualities for an intended purpose. Some of those performance parameters are standardized by independent authorities, others are defined by certain manufacturers of hygiene products for their individual needs only. Actually, both types of performance parameters are of importance within the real-life SAP market. Besides that, the patent literature is jam-packed with parameters which do not have any impact outside a certain patent right at all.

For the present invention, the following performance parameters are relevant, and were determined as follows:

### Centrifuge Retention Capacity (CRC)

The centrifuge retention capacity refers to the fluid retention capacity without pressure. The method WSP 241.2 (05) recommended by EDANA (European Disposables and Nonwovens Associations) was applied.

### Absorption Against Pressure (AAP)

The absorption against pressure refers to the capacity of absorbing urine when subjected to an external pressure. The AAP was measured at a pressure of 4.83 kPa (which is equal to 0.7 psi). The method WSP 242.2 (05) recommended by EDANA (European Disposables and Nonwovens Associations) was applied.

### Saline Flow Conductivity (SFC)

The saline flow conductivity refers to the ability of a gel to transfer fluids once a first contact with body fluid has occurred. The test method is disclosed in the International Publication WO-A-95/26209, pages 69 to 75, although 1.5 g of the to-be-investigated superabsorbent was used instead of 0.9 g.

### Free Swell Rate (FSR)

This parameter relates to the swelling speed of a water-absorbent polymer. 1.00 g (=W1) of the dry water-absorbing material is weighed into a 25 ml glass beaker and is uniformly distributed on the base of the glass beaker. 20 ml of a 0.9 wt. % sodium chloride aqueous solution are then dispensed into a second glass beaker, the contents of this beaker are rapidly added to the first beaker and a stopwatch is started. As soon as the last drop of salt solution is absorbed, confirmed by the disappearance of the reflection on the liquid surface, the stopwatch is stopped. The exact amount of liquid poured from the second beaker and absorbed by the polymer in the first beaker is accurately determined by weighing back the second beaker (=W2). The time needed for the absorption, which was measured with the stopwatch, is denoted t. The disappearance of the last drop of liquid on the surface is defined as time t. The free swell rate (FSR) is calculated as follows:

FSR[g·g⁻¹·s⁻¹]=W2/(W1·t)

When the moisture content of the investigated material is more than 3 wt. %, however, the weight W1 must be corrected for this moisture content. The unit of the FSR is g·g⁻¹·s⁻¹.

### Vortex Time

The vortex time refers to the time in seconds until the vortex disappeared after adding 2 g of a water-absorbent polymer to 50 mL of physiological saline solution and then stirring the mixture at 600 rpm. A 100 mL glass beaker (Pyrex #1060-100 or Fisher Brand #FB-102-100) was used together with a magnetic stirring rod (size 38.4 mm×8 mm). The saline temperature was set to be between 24 °C and 25 °C and the temperature of the room was above 21 °C. The humidity was set to be within the limitations of EDANA Standard Test WSP 241.2 (05).

Invention shall now more elucidated by means of following examples.

### EXAMPLES

### Preparation of a Dried Crosslinked Water-Absorbing Polyglutamic Acid (Salt) (Table 1)

### Preparation of an Aqueous Crosslinker Solution

1,000 g deionized water and 10 g of a diglycidyl ether type crosslinker were carefully mixed in a beaker with the help of a magnetic stirrer at room temperature. The used crosslinkers were polyethylene glycol diglycidyl ether (poly-EGDGE) or 1,4-butandiol diglycidyl ether (BDDGE) which were used in an amount of 2 wt. % based on the amount of the non-crosslinked polyglutamic acid (salt). More information is given in the Table 1 below.

### Batch Crosslinking in the Kneader Reactor

The crosslinking reactions were conducted in a single shaft kneader reactor of the type Discotherm Batch, DTB1.5, obtained from List AG, Switzerland, with a total volume of 3.1 liters. The reactor is equipped with a jacket for heating and cooling, a vacuum system, a nitrogen supply system, and a reactor control unit.

The water/crosslinker solution prepared as described above, was filled into the reactor through the dome and thereafter 500 g γ-PGA (obtained from Lubon Industry Co. Ltd., China, MW=700,000 Dalton; or obtained from Bonding Chemical, USA, MW=1,100,000 Dalton; see Table 1 below) in solid form is added under full speed agitation (65 rpm). The reactor was previously heated up to more than 110 °C (set temperature of the heating device: 120 °C). The temperature was measured in the jacket of the kneader reactor. The pressure of the vessel is the atmospheric pressure. The reactor is kept open and at the previous mentioned temperature over the entire reaction.

The agitation is set to the maximum (100% of the potentiometer, 65 rpm) for 20 minutes to allow the PGA to dissolve completely and the mixture to reach the wished temperature. After this, the agitator is set to 40% which is 24 rpm, for additional 10 minutes. Thereafter, the endplate of the reactor is removed to discharge the free flowing and granulated gel from the reactor. The obtained polymer is further processed.

The granulated gel obtained from the crosslinking in the DTB 1.5 I reactor is extruded in a kitchen type meat mincer (Mado Kuchen Fleischwolf MEW 710-R70) equipped with a die plate of 8 mm thickness having 45 holes each with a diameter of 6 mm.

### Drying the Crosslinked Polyglutamic Acid (Salt)-Gel

Gel drying was performed in a batch-operated Fluidized Bed Drier, type CTL (delivered by Allgaier-Werke KG, Uhingen, Germany). This drier is equipped with a conically shaped fluidization chamber having on the bottom side the same plate as the plate belt dryers of the plant, namely perforated stainless steel plates with 20×3 mm holes, diameter of 20 cm, a ventilator, an air heater, a fresh air and an exhaust air filter with automatic de-dusting and a control box. The air stream is flowing bottom-up and is not circulated.

A portion of 600 to 900 grams of the extruded gel is then placed in the fluidization chamber of the dryer. The gel to be dried is placed on the plate, the gel bed having a thickness of about 5 cm to 8 cm and is dried in the hot air stream at 5.5 m/s air inlet velocity at 130 °C or 140 °C for 15 minutes or 20 minutes, respectively. After 10 minutes the pre-dried material is turned by 180° to allow a homogeneous drying on both sides for further 5 or 10 minutes. The dry polymer obtained is ground in a Bauermeister lab roll mill and sieved in a Retsch sieve tower equipped with the sieves having mesh sizes of 850 and 150 microns, obtaining the particle size fraction for analysis of the precursor superabsorber.

### Heat Treating the Dried Crosslinked Water-Absorbing Polyglutamic Acid (Salt) (Table 2)

The dried crosslinked water-absorbing polyglutamic acid (salt) obtained after drying, grinding and sieving was heat treated in the presence of a facilitator improve its properties, see Table 2. To prepare the surface-crosslinking solution 0.3 wt. % to 1.5 wt. % crosslinker was dissolved in 3 wt. % demineralized water, each based on 100 g of dried crosslinked water-absorbing polyglutamic acid (salt).

As crosslinker, ethylene glycol diglycidyl ether (EGDGE) or 1,4-butandiol diglycidyl ether (BDDGE) were employed. Additives such as isopropanol, aluminum lactate were used. Also, water was employed as facilitator.

60 g of the dried crosslinked water-absorbing polyglutamic acid (salt) was coated in a household Krupps blender cup with the aforementioned amount of surface-crosslinking solution by means of a syringe with 0.4 mm cannula while strongly stirring with a Krupps 3 Mix 7000 mixer and stirred for another minute.

Details on used facilitators, amounts and reaction conditions are provided in Table 2 below.

The coated precursor was divided into 3 portions and heated in a circulating air-drying oven Heraeus UT6120 for 20 to 60 minutes at 150 °C.

The surface crosslinked polymers thus obtained were sieved with a Retsch sieve having a mesh size of 850 microns to remove agglomerates formed by the coating.

### Results

The performance data of water-absorbing polyglutamic acid (salt) prepared are shown in the tables.

TABLE 1
Data of the dried crosslinked polyglutamic acid (salt)-gel.

| | | | | | | | | AAP | | | |
|---|---|---|---|---|---|---|---|---|---|---|---|
| Lab | | | Temperature | Drying | Drying | Gel | | 0.7 | SFC | | FSR |
| ID | γ-PGA | crosslinker | Heat jacket | temp | time | amount | CRC | psi | [10⁻⁷ cm³ · | Vortex | [g · |
| [#] | [Da] | [—] | [°C] | [°C] | [min] | [g] | [g/g] | [g/g] | s · g⁻¹] | [s] | g⁻¹ · s⁻¹] |
| 9.1 | 1,100,00 | p-EGDGE | 105 | 140 | 15 | 600 | 25.8 | 16.1 | 8 | 19 | 0.82 |
| 9.2 | 1,100,00 | p-EGDGE | 105 | 130 | 15 | 600 | 26.3 | 14.2 | 11 | 29 | 0.69 |
| 17.1 | 700,000 | p-EGDGE | 105 | 140 | 15 | 900 | 26.4 | 16.1 | — | — | — |
| 17.2 | 700,000 | p-EGDGE | 105 | 140 | 20 | 900 | 26.9 | 15.8 | 7 | 20 | 0.74 |
| 22.1 | 1,100,00 | p-EGDGE | 115 | 130 | 15 | 600 | 32.9 | 9.1 | — | — | — |
| 23.1 | 1,100,00 | p-EGDGE | 115 | 130 | 15 | 600 | 25.4 | 21.1 | — | — | — |
| 23.2 | 1,100,00 | 1,4-BDDGE | 115 | 130 | 15 | 600 | 25.6 | 21.5 | — | — | — |
| 24 | 1,100,00 | 1,4-BDDGE | 115 | 130 | 15 | 700 | 27.4 | 21.1 | — | — | — |
| 27.2 | 700,000 | 1,4-BDDGE | 115 | 130 | 15 | 600 | 24.2 | 21.4 | — | — | — |
| 31.1 | 1,100,00 | 1,4-BDDGE | 105 | 140 | 15 | 650 | 23.0 | 22.5 | — | — | — |
| 33.1 | 1,100,00 | 1,4-BDDGE | 105 | 140 | 15 | 650 | 23.5 | 22.2 | — | — | — |
| 34.2 | 1,100,00 | 1,4-BDDGE | 105 | 140 | 15 | 650 | 25.3 | 21.8 | — | — | — |

TABLE 2
Data of the water-absorbing polyglutamic acid (salt) after heat treatment in the presence of a facilitator.

| | | Amount | | | | surface crosslinking | | AAP | SFC | | |
|---|---|---|---|---|---|---|---|---|---|---|---|
| Lab | Amount | 1,4- | Amount | Amount | Aluminum | condition (duration | | 0.7 | [10⁻⁷ | | FSR |
| ID | EGDGE | BDDGE | water | Isopropanol | lactate | and temperature) | CRC | psi | cm³ · | Vortex | [g· |
| [#] | [wt-%] | [wt-%] | [wt-%] | [wt-%] | [wt-%] | [min/°C] | [g/g] | [g/g] | s · g^{-1]} | [s] | g⁻¹ · s⁻¹] |
| 9.1-A | 0.5 | — | 3 | 1 | — | 20/150 | 25.8 | 21.1 | 26 | 28 | 0.78 |
| 9.1-B | 0.5 | — | 3 | 1 | — | 40/150 | 27.3 | 21.2 | — | — | — |
| 9.1-C | 0.5 | — | 3 | 1 | — | 60/150 | 28.0 | 20.9 | 19 | 25 | 0.79 |
| 9.2-A | — | 0.8 | 3 | — | — | 20/150 | 26.2 | 21.5 | 27 | 30 | 0.70 |
| 9.2-B | — | 0.8 | 3 | — | — | 40/150 | 27.2 | 21.5 | — | — | — |
| 9.2-C | — | 0.8 | 3 | — | — | 60/150 | 28.8 | 21.2 | 20 | 29 | 0.73 |
| 17.1-D | — | 0.8 | 3 | — | 0.3 | 20/150 | 26.6 | 19.1 | 25 | — | 0.75 |
| 17.1-E | — | 0.8 | 3 | — | 0.3 | 40/150 | 26.3 | 19.0 | — | 20 | — |
| 17.1-F | — | 0.8 | 3 | — | 0.3 | 60/150 | 27.2 | 19.3 | 27 | — | 0.64 |
| 17.2-A | — | 0.8 | 3 | — | — | 20/150 | 27.0 | 19.2 | — | 30 | 0.73 |
| 17.2-B | — | 0.8 | 3 | — | — | 40/150 | 28.7 | 19.1 | — | — | — |
| 17.2-C | — | 0.8 | 3 | — | — | 60/150 | 28.4 | 18.8 | — | — | 0.68 |
| 22.1-A | — | 0.8 | 3 | — | — | 20/150 | 38.1 | 11.3 | | 24 | 0.80 |
| 22.1-B | — | 0.8 | 3 | — | — | 40/150 | 43.7 | 10.2 | — | — | — |
| 22.1-C | — | 0.8 | 3 | — | — | 60/150 | 45.8 | 9.6 | | 22 | 0.88 |
| 23.1-A | — | — | 3 | — | — | 20/150 | 29.4 | 23.2 | 38 | 19 | 1.02 |
| 23.1-B | — | — | 3 | — | — | 40/150 | 32.2 | 22.8 | — | — | — |
| 23.1-C | — | — | 3 | — | — | 60/150 | 33.6 | 23.3 | 17 | 19 | 1.11 |
| 23.2-C1 | — | — | 3 | — | — | 60/150 | 31.2 | 22.4 | — | — | — |
| 23.2-C2 | — | — | 3 | — | — | 60/150 | 32.2 | 23.2 | — | — | — |
| 23.2-C1 + 2 | — | — | 3 | — | — | 60/150 | 31.7 | 22.8 | 26 | 15 | 1.00 |
| 24-A | — | — | 3 | — | — | 20/150 | 32.7 | 20.9 | 14 | 24 | 0.86 |
| 24-B | — | — | 3 | — | — | 40/150 | 37.3 | 19.4 | — | — | — |
| 24-C | — | — | 3 | — | — | 60/150 | 38.6 | 16.6 | — | — | — |
| 24-D | — | 0.8 | 3 | — | — | 20/150 | 32.5 | 21.7 | 14 | 21 | 0.90 |
| 24-E | — | 0.8 | 3 | — | — | 40/150 | 36.6 | 20.2 | — | — | — |
| 24-F | — | 0.8 | 3 | — | — | 60/150 | 38.0 | 17.8 | — | — | — |
| 27.2-A | — | — | 3 | — | — | 20/150 | 27.0 | 23.2 | 65 | 23 | 0.77 |
| 27.2-B | — | — | 3 | — | — | 40/150 | 28.3 | 22.8 | 54 | 21 | 0.80 |
| 27.2-C | — | — | 3 | — | — | 60/150 | 28.9 | 23.0 | 48 | 24 | 0.81 |
| 31.1-A | — | — | 3 | — | — | 20/150 | 24.0 | 24.0 | — | — | — |
| 31.1-B | — | — | 3 | — | — | 40/150 | 24.8 | 24.8 | — | — | — |
| 31.1-C | — | — | 3 | — | — | 60/150 | 23.7 | 23.7 | — | 20 | — |
| 33.1-A | — | — | 3 | — | — | 20/150 | 24.3 | 24.3 | — | — | — |
| 33.1-B | — | — | 3 | — | — | 40/150 | 24.5 | 24.5 | — | — | — |
| 33.1-C | — | — | 3 | — | — | 60/150 | 22.9 | 22.9 | — | — | — |
| 34.2-C | — | — | 3 | — | — | 60/150 | 22.1 | 22.1 | — | 21 | — |

When water is the only facilitator used the water-absorbing polyglutamic acid (salt) shows good simultaneous performance regarding AAP and CRC. The overall performance is comparable to the performance of acrylic acid based superabsorbent polymers but the polyglutamic acid (salt) based polymers are environmentally friendly.

1. A method for producing a water-absorbing polyglutamic acid (salt), comprising the method steps of:
i) providing an aqueous mixture of a non-crosslinked polyglutamic acid (salt) and at least one crosslinker,
ii) crosslinking the non-crosslinked polyglutamic acid (salt) to obtain a crosslinked polyglutamic acid (salt)-gel,
iii) drying the crosslinked polyglutamic acid (salt)-gel to obtain a dried crosslinked water-absorbing polyglutamic acid (salt),
iv) heat treating the dried crosslinked water-absorbing polyglutamic acid (salt) in presence of at least one facilitator to obtain the water-absorbing polyglutamic acid (salt).

2. The method according to claim 1, characterized in, that the non-crosslinked polyglutamic acid (salt) has a molecular weight of 200,000 Dalton to 3,000,000 Dalton.

3. The method according to claim 1, characterized in that the crosslinker is selected from the group consisting of glycidyl ether such as diglycidylether, triglycidylether, polyglycidylether containing 3 or more epoxy groups, diglycerol tetraglycidyl ether, dipentaerythritol tetraglycidyl ether, pentaerythritol polyglycidyl ether, sorbitol polyglycidylether, isosorbide glycidyl ethers, polyglycerol-3-glydidyl ether, or other aliphatic polyfunctional epoxides, 1,4-butanediol diglycidyl ether, 1,6-hexanediol diglycidyl ether, propylene glycol diglycidyl ether, di propylene glycol diglycidyl ether, trimethylolpropane triglycidyl ether, poly propylene glycol diglycidyl ether, polyglycidyl ethers of alkanepolyols, polyglycidyl ethers of poly(alkylene glycols), biobased sorbitol glycidyl ether, cyclic and aromatic poly glycidylethers, or a combination of more than one crosslinker of the class of glycidyl ether, the class of carbodiimide such as 1-ethyl-3-(3-dimethylaminopropyl) carbodiimide hydrochloride, dicyclohexyl carbodiimide or other carbodiimides containing compounds or a combination of carbodiimide functionality containing crosslinker with other crosslinkers e.g. crosslinker of the class of glycidyl ethers saccharides such as glucose, maltotriose or cyclodextrin in the presence of water-soluble carbodiimide containing compound, other crosslinkers such as water-soluble chitosan, polyethylene glycol or other organic poly alcohols, aryl azide or diazirine or other photoreactive chemical hetero bi-functional crosslinking agents acting as receptor-ligand-interaction complexes by a 2-step activation, ethylene glycol diglycidyl ether, polyethylene glycol diglycidyl ether and 1,4-butanediol diglycidyl ether, and preferably from the group consisting of ethylene glycol diglycidyl ether, polyethylene glycol diglycidyl ether and 1,4-butanediol diglycidyl ether.

4. The method according to claim 3, characterized in that the at least one crosslinker is selected from the group consisting of ethylene glycol diglycidyl ether, poly(ethylenglycol) diglycidylether and 1,4-butanediol diglycidyl ether.

5. The method according to claim 1, characterized in that
the aqueous mixture comprises:
a) 10.0 wt. % to 50.0 wt. %, preferably 20.0 wt. % to 40.0 wt. %, and more preferably 30.0 wt. % polyglutamic acid (salt) based on the total weight of the aqueous mixture,
b) 0.1 wt. % to 4.0 wt. %, preferably 0.3 wt. % to 3.0 wt. %, and more preferably 0.5 wt. % to 2.0 wt. % of the at least one crosslinker based on the total weight of the aqueous mixture,
c) 0.0 wt. % to 10.0 wt. %, preferably 0.0 wt. % to 5.0 wt. %, and more preferably 0.1 to 2.5 wt. % of a water-soluble polymer, and
d) 30.0 wt. % to 90.0 wt. %, preferably 50.0 wt. % to 80.0 wt. %, and more preferably 60.0 wt. % to 70.0 wt. % water.

6. The method according to claim 1, characterized in that
the aqueous mixture comprises 25.0 wt. % to 50.0 wt. %, preferably 30.0 wt. % to 40.0 wt. %, and more preferably 33.3 wt. % of a mixture comprising the polyglutamic acid (salt) and the at least one crosslinker based on the total weight of the aqueous mixture.

7. The method according to claim 1, characterized in that
step ii) is conducted within 10 min to 50 min, preferably within 20 min to 40 min, and more preferably within 30 min; and/or
step ii) is conducted under atmospheric pressure; and/or
step ii) is conducted under agitation; and/or
step ii) is conducted in a kneader reactor.

8. The method according to claim 7, characterized in that
step ii) comprises a first stage and a second stage, and the time duration of the first stage is between 50% to 82%, preferably 58% to 76%, and more preferably 66% of the total time duration of step ii), and the first stage is conducted under maximum agitation and the second stage at 20% to 60%, preferably 30% to 50% and more preferably 40% of the maximum agitation.

9. The method according to claim 1, characterized in that
the crosslinked polyglutamic acid (salt)-gel is disintegrated before drying.

10. The method according to claim 9, characterized in that disintegrating is performed by an extruder or chopper or mincer.

11. The method according to claim 1, characterized in that
step iii) is conducted using a bed drier, a belt drier, a fluidized bed drier, a microwave or a convection drier.

12. The method according to claim 1, characterized in that
step iii) is conducted for 5 min to 15 min, preferably for 10 min at a temperature of between 100 °C to 160 °C, preferably between 115 °C to 145 °C, and more preferably at a temperature of 130 °C.

13. The method according to claim 1, characterized in that
the dried polyglutamic acid (salt)-gel is subject to a grinding step and a sieving step prior to step iv).

14. The method according to claim 13, characterized in that
a sieving apparatus is used in the sieving step, wherein the sieving apparatus has at least two sieves, the first sieve has a mesh size of 850 μm and the second mesh size of 150 μm.

15. The method according to claim 1, characterized in that
in step iv) at least one facilitator is used selected from the group consisting surface crosslinker, water and an additive.

16. The method according to claim 15, characterized in that
the at least one facilitator used in step iv) is selected from the group consisting of ethylene glycol diglycidyl ether, 1,4-butanediol diglycidyl ether, and water.

17. The method according to claim 15, characterized in that
0.10 wt. % to 2.00 wt. %, preferably 0.25 wt. % to 1.40 wt. %, and more preferably 0.50 wt. % to 0.80 wt. % of the surface crosslinker is used based on the total amount of the dried water-absorbing polyglutamic acid (salt).

18. The method according to claim 1, characterized in that the facilitator is water.

19. The method according to claim 18, characterized in that 1.0 wt. % to 5.0 wt. %, preferably 2.0 wt. % to 4.0 wt. %, and more preferably 3.0 wt. % water is used based on the total amount of the dried water-absorbing polyglutamic acid (salt).

20. A water-absorbing polyglutamic acid (salt) obtainable by a method according to claim 1.

21. A water-absorbing polyglutamic acid (salt), characterized in that said water-absorbing polyglutamic acid (salt) possesses at least one of the following features:
an absorption against pressure of 4.83 kPa (0.7 psi) of more than 9 g/g and less than 30 g/g or less than 26 g/g, preferably less than 25 g/g, as measured as defined in the description;
a saline flow conductivity of more than 0 and less than 150×10⁻⁷ cm³·s·g⁻¹, preferably more than 10 and less than 100×10⁻⁷·cm³·s·g⁻¹ as measured as defined in the description;
a centrifugal retention capacity of more than 20 and less than 46 g/g, preferably more than 25 and less than 42 g/g as measured as defined in the description;
a free swell rate of more than 0.5 and less than 1.5 g·g⁻¹·s⁻¹, preferably 0.7 and less than 1.2 g·g⁻¹·s⁻¹ as measured as defined in the description;
a Vortex of less than 50 and more than 10 seconds, preferably less than 30 and more than 15 seconds as measured as defined in the description.

22. An article comprising the water-absorbing polyglutamic acid (salt) according to claim 20.

23. The article according to claim 22, characterized in that the article is or comprises a diaper, hygiene article, a sanitary towel, a napkin, a wound covering.
